(19) United States
(12) Patent Application Publication
(10) Pub. No.: US 2025/0213403 A1
(43) Pub. Date: Jul. 3, 2025

(54) PERSONNEL TRANSPORTATION POD

(71) Applicant: SABRN TECH PTY LTD, Craigburn Farm (AU)

(72) Inventor: Abhilash Chandra, Craigburn Farm (AU)

(21) Appl. No.: 18/849,434

(22) PCT Filed: Mar. 24, 2023
(86) PCT No.: PCT/AU2023/050215

§ 371 Date: Sep. 20, 2024

(30) Foreign Application Priority Data
Mar. 25, 2022 (AU) 2022900756

(51) Int. Cl. A61G 3/00; A61B 34/30; A61G 1/013; A61G 1/044; A61G 3/08; A61G 10/02

(52) U.S. Cl. CPC A61G 3/001; A61B 34/30; A61G 1/013; A61G 1/044; A61G 3/0816; A61G 10/02

(57) ABSTRACT

A personnel transportation pod for transporting, remote monitoring of, and/or providing medical support to at least one individual, the pod comprising: an airtight structure comprising walls forming an enclosed interior and an exposed exterior, wherein at least one of the walls comprise at least one opening for providing a passage for the individual from the exterior of the pod to the interior and vice versa, wherein the opening is sealable by a hatch; a connection means on the exterior of the pod, wherein the connection means enables the pod to be connected to a vehicle; a remote monitoring system comprising a monitoring means; a medical support system comprising life support equipment and at least one remotely operable robot capable of manipulating equipment and/or each individual; and a communication system, wherein in use, the communication system enables remote monitoring of each individual within the pod and remote utilisation of each robot to treat each individual.

### PRIORITY DOCUMENT

The present application claims priority from Australian Provisional Patent Application No. 2022900756 titled “PERSONNEL TRANSPORTATION POD” and filed on 25 Mar. 2022, the content of which is hereby incorporated by reference in its entirety.

### TECHNICAL FIELD

The present disclosure relates to the field of personnel transportation pods. In particular, the present disclosure relates to a personnel transportation pod for transporting one or more individual from a first location to a second location.

### BACKGROUND

Personnel in industries that operate in isolated and/or hazardous locations, such as military or mining, can face life-threatening emergencies. These emergencies can vary widely in magnitude. The austere terrain or injuries to the individual may make evacuation difficult (e.g. in an underground mining accident or a combat environment). The inherent locations and working conditions of these industries often correlate with the lack of medically-trained personnel to treat such emergencies. Instead, an on-site (para) medic or nurse works to stabilize the injured patient until they are in a transportable condition to be extricated to a hospital or other suitable medical facility where definitive surgical or medical treatment can be administered. The isolation and remoteness of these industries requires the (para) medic or nurse to initiate telehealth communications with a medically qualified professional for support in treating the life-threatening emergency. Current medical extraction systems from these environments have several significant deficiencies and can compromise the clinical outcome of the casualty.

The personnel extraction process from these isolated and remote locations can be improved significantly with more effective communication systems and incorporation of improved diagnostic and therapeutic systems within the extraction process at the site of injury.

Remote-controlled or autonomous systems may have particular use in applications where transportation is required for one or more injured or unwell individual, which may be located or stranded in an unfamiliar and/or hostile location. A solution may be the use of an enclosed ‘pod’ or ‘capsule’ to transport individuals using remote-controlled or autonomous systems. In particular, specifically designed, equipped and integrated ‘pods’ or ‘capsules’ may have the capability of ‘bringing the hospital to the individual’, especially when the individual is located in extreme austere environments (e.g. hostile terrains, chemical-biological-radiological-nuclear (CBRN), marine, submarine and space environments). However, such integrated transportation and extraction systems do not exist, and could be desirable by defence, remote/dangerous and humanitarian industries.

These ‘pods’ or ‘capsules’ can be classified as ‘make-shift’, ‘mobile’, ‘temporary’, or ‘transportable’, to serve as an intermediary or ‘triage’ role set up and located near the unfamiliar and/or hostile location. These ‘pods’ or ‘capsules’ can be utilised as clinical diagnostic and therapeutic facilities that can be set up, located, or designated to isolated populations, vulnerable populations, populations with overwhelmed traditional hospitals (e.g. ambulance ‘ramping’ at Emergency Departments), or specialty industries. Currently, most existing ‘mobile’ clinical facilities require multiple components or individual systems that require configuration and/or construction in order to be operable.

A medical isolation and containment facility is one such type of healthcare facility. These are designed to provide the initial point-of-care for patients that are isolated, or not immediately transportable to a hospital or other suitable medical facility. Often, these patients are required to be cared for in a manner to limit transmission of infections agents. These facilities often accommodate multiple individuals, and may comprise one or more treatment room requiring a large space in which they are to be set up. Furthermore, these facilities are not mobile with patients or practitioners therein, and have limited environments in which they can be functional.

It is against this background and the problems and difficulties associated therewith, that the present disclosure has been developed.

Throughout this disclosure, the term ‘pod’ should be understood to mean an elongated vessel capable of housing one or more person therein. When combined with the defining terms ‘personnel transportation’, or ‘patient retrieval’, or ‘life sustaining’, or ‘personnel housing’ the pod of the present disclosure should be understood as a vessel designed for use in various scenarios, including for sustaining life of the one or more person therein, being a mobile/portable transportable critical care unit for the one or more person therein, and being capable of transporting individuals from a first location to a second location. Accordingly, the pod of the present disclosure could be understood as any one or more of a survival pod, a rescue pod, a medical pod, a self-contained pod, a transportable pod, or the like.

Additionally, throughout this disclosure, the term ‘individual’, ‘patient’, ‘person’, ‘personnel’ and other similar terms, should be understood to mean one or more human being and may be used interchangeably.

### SUMMARY

Embodiments of the present disclosure relate to a personnel transportation pod for transporting and protecting of one or more individual therein from an isolated or hazardous location to a safe location such as a hospital. The one or more individual may include one or more injured individual and one or more medical personnel (such as a paramedic, nurse, doctor or surgeon), wherein the injured individual(s) require medical attention not sufficiently available at the isolated or hazardous location. The personnel transportation pod comprises an airtight and secure structure capable of protecting the injured individual(s) therein from an external hazardous environment, and one or more means that enable remote monitoring, assessment, and treatment of the injured individual(s) therein. The structure of the pod comprising a connection means that enables the pod to be connected to any manned or unmanned vehicles that enable transportation thereof (such as vehicles that are locally or remotely operated and are capable of transporting the system in land, air, sea, and space) to the safe location.

In one embodiment, the present disclosure relates to a personnel transportation pod that is configured to be connectable to any manned or unmanned vehicle for the transportation of one or more individual within the pod from a first location (which may be an isolated or hazardous location) to a second location (which may be a safe location, such as a hospital). The personnel transportation pod comprising one or more system particularly designed to remotely monitor and provide life support to the one or more individual within the pod. The pod may also comprise an exposed exterior with an external surface that is resistant to environmental conditions, such that the pod is able to ensure that the individual(s) therein survive in an austere or hostile environment.

According to a first aspect of the present disclosure, there is provided a personnel transportation pod for transporting, remote monitoring of, and/or providing medical support to at least one individual, the personnel transportation pod comprising: an airtight structure comprising walls forming an enclosed interior and an exposed exterior, wherein at least one of the walls comprise at least one opening for providing a passage for the individual from the exterior of the pod to the interior and vice versa, wherein the opening is sealable by a hatch; a connection means on the exterior of the pod, wherein the connection means enables the pod to be connected to a vehicle; a remote monitoring system comprising a monitoring means; a medical support system comprising life support equipment and at least one remotely operable robot capable of manipulating equipment and/or each individual; and a communication system wherein in use, the communication system enables remote monitoring of each individual within the pod and remote utilisation of each robot to treat each individual.

In one embodiment, the airtight structure of the pod seals the enclosed interior from an external environment. In this embodiment, the external environment may comprise hazards such as hazardous gas, dust, extreme heat or cold, submersion under water, lack of breathable air, chemical contamination,-biological contamination, radiological contamination,-nuclear contamination, low pressure environments, high pressure environments, space, or the like. Additionally in this embodiment, the airtight structure may comprise one or more vent coupled to a decompression system, wherein in use, the decompression system is utilised for the increase or decrease in ambient pressure within the enclosed interior of the pod.

In one embodiment, the pod further comprises at least one integrated support stretcher in the interior of the pod.

In one embodiment, the support stretcher is removable from the interior of the pod to the exterior via the hatch, and vice versa, to assist in accommodating the individual(s) within the pod.

In one embodiment, the pod further comprises at least one integrated seat in the interior of the pod.

In one embodiment, the support stretcher comprises a segmented base, wherein each segment of the stretcher is removable and comprises a support strap to assist in securing individual(s) thereto.

In one embodiment, the segmented base of the support stretcher enables reconfiguration of the stretcher into a seat. In this embodiment, the segmented base enables the support stretcher to be folded and stowed.

In one embodiment, the exposed exterior of the pod comprises an external surface that is resistant to environmental conditions, and the enclosed interior of the pod comprises an internal surface that is at least partially separated from the external surface by insulation.

In one embodiment, the external and internal surfaces are separated by thermal insulation capable of maintaining suitable conditions within the interior of the pod to sustain the individuals. In another embodiment, the external and internal surfaces are further separated by additional reinforcement layers to aid in providing protection to the pod and the individuals therein.

In one embodiment, the external surface is sufficiently reinforced enabling the pod to withstand impact.

In one embodiment, the external surface is any one or more of Kevlar, Twaron, graphene, multi-walled carbon nanotubes, or similar material(s) capable of providing protection and heat-resistance to the pod.

In one embodiment, the pod is sized and shaped to house additional individuals, such as a medical practitioner, for providing life support to the other individual(s).

In one embodiment, the connection means enables the pod to be connected to an external uninhabited ground vehicle (UGV), wherein the UGV is remotely operable and capable of transporting and retrieving the pod from the first location, such as an isolated or hazardous location, to the second location, such as a safe location.

In one embodiment, the connection means comprises stabilizing features, such as electromagnetic or air cushions, whereby when the pod is connected to the UGV or the vehicle, the stabilizing features improve the stability of the pod with respect to the UGV or vehicle.

In one embodiment, the connection means enables the pod to be connected to a mobile platform, whereby the mobile platform is capable of supporting and transporting the pod.

In one embodiment, the mobile platform comprises a dock for receiving the connection means of the pod, a plurality of wheels and/or tracks on an underside of the dock to enable transport of the platform, and one or more tethering mechanism enabling the mobile platform to be connected to a UGV or a vehicle.

In one embodiment, the medical support system further comprises an oxygen supply system for providing oxygen to the interior of the pod.

In one embodiment, the oxygen supply system comprises oxygen tanks and an oxygen generator.

In one embodiment, at least one hatch maintains the airtight structure.

In one embodiment, the vehicle is any one of a manned or unmanned vehicle, whereby the manned or unmanned vehicle enables the transport of the pod and the individual(s) contained therein to be transported from the first location to the second location.

In one embodiment, the remote monitoring system further comprises an environmental monitoring means for monitoring the interior and/or exterior atmosphere of the pod.

In one embodiment, the environmental monitoring means monitors the exterior atmosphere of the pod to ensure that the interior of the pod is suitably maintained to enable survivability of the individual(s) therein.

In one embodiment, the environmental monitoring means monitors the exterior atmosphere of the pod to ensure that the pod is operable in austere or hostile environments.

In one embodiment, the life support equipment comprises medical and surgical equipment that is capable of being manipulated by the at least one remotely operable robot.

In one embodiment, the pod further comprises a power system comprising one or more means for providing power to the pod.

In one embodiment, the one or more means for providing power to the pod transfers power derived from a source of renewable energy to the pod.

In one embodiment, the source of renewable energy is any one of solar or hydrogen energy stored in one or more battery or other storage medium attached to the interior of the pod.

In one embodiment, the pod further comprises at least one connection port for connecting the pod to an external power source to charge the power system.

In one embodiment, at least one connection port further connects the pod to additional communication systems.

In one embodiment, the communication system comprises a multi-modal communication means comprising one or more of video, voice and immersive technology-mediated communication means that enable a remote user, such as a medical professional, to virtually monitor the individuals within the pod.

According to a second aspect, there is provided a personnel rescue system for transporting, remote monitoring of, and/or providing medical support to at least one individual, the system comprising: a pod and at least one modular stretcher, wherein the pod comprises: an airtight structure having an enclosed interior, an exposed exterior, and at least one opening providing for providing a passage between the exterior and the interior and vice versa, wherein the opening is sealable by a hatch; a connection means on the exterior of the pod, wherein the connection means enables the pod to be connected to a vehicle; a remote monitoring system for monitoring the individual; a medical support system for stabilising, sustaining and providing medical care to the individual; and a communication system, wherein in use, the communication system enables remote monitoring of each individual within the pod and remote utilisation of the medical support system; and wherein, in use, the at least one modular stretcher transports the individual from a location exterior of the pod to the enclosed interior of the pod, and the remote monitoring, medical support and communication systems is utilised to monitor, support and communicate with the individual within the pod.

### BRIEF DESCRIPTION OF DRAWINGS

Embodiments of the present disclosure will be discussed with reference to the accompanying drawings wherein:

FIG. 1 is a perspective view illustrating an embodiment of the personnel transportation pod;

FIG. 2 is a perspective view of the personnel transportation pod of FIG. 1 connected to a mobile platform, wherein a hatch or door of the pod is in an open position;

FIG. 3 is a schematic illustration of the personnel transportation pod, wherein the mobile platform is further connected to an external uninhabited ground vehicle;

FIG. 4 is an alternate perspective view illustrating an embodiment of the personnel transportation pod, wherein the hatch or door of the pod is in a closed position;

FIG. 5 is a perspective view of the personnel transportation pod of FIG. 4 with the hatches of the pod removed to illustrate the internal components of the pod;

FIG. 6 is a schematic planar sectional view of the pod of FIGS. 4 and 5;

FIG. 7 is a further schematic sectional view of the personnel transportation pod of FIG. 6, illustrating the support stretcher comprising a segmented base, wherein one section of the segmented base is raised;

FIG. 8 is a perspective view of FIG. 6;

FIG. 9 is a schematic sectional front view of the personnel transportation pod of any one of FIGS. 4 to 8;

FIG. 10 is a schematic front view of the personnel transportation pod of FIG. 9, illustrating an embodiment of the pod comprising two hatches in an open position;

FIG. 11 is a perspective view of an embodiment of a modular stretcher that is capable of being received within and connected to the pod of any one of the Figures;

FIG. 12 is a schematic view of the modular stretcher of FIG. 11 with its doors in an open position;

FIG. 13 is a perspective view of the modular stretcher of FIGS. 11 and 12 in an unfolded position;

FIG. 14 is a top view of the modular stretcher of FIG. 13;

FIG. 15 is a perspective schematic view of an embodiment of a rescue system; and

FIG. 16 is a topographical view of the rescue system of FIG. 15.

In the following description, like reference characters designate like or corresponding parts throughout the figures.

### DESCRIPTION OF EMBODIMENTS

Referring to any one of the Figures, there is disclosed a personnel transportation pod 100 that is configured to be connectable to any manned or unmanned vehicle capable of transporting the pod 100, and one or more individual within the pod 100, from a first location (which may be an unfamiliar, isolated, hostile and/or hazardous location) to a second location (which may be a safe location, such as a hospital, clinic, base camp or the like). The personnel transportation pod 100 comprises one or more system particularly designed to allow for the remote monitoring of and provide medical/life support to the one or more individual, until that individual is transported from the first location to the second location.

Before continuing further, it is to be noted that references to the one or more individual in the personnel transportation pod 100 may include one or more injured individual and one or more medical personnel (such as a paramedic, nurse, doctor or surgeon), wherein the injured individual(s) require medical attention not sufficiently available at the first location. It is also to be noted that in the description below, it will be understood that the personnel transportation pod 100 is a ‘critical care unit’ (also may be referred to as an ‘intensive care unit’ or ‘intensive therapy unit’) that is capable of acting as a transportable health care facility that enables medical personnel to care for injured personnel therein, as the injured personnel is transported from the first location to the second location.

A preferred embodiment of the personnel transportation pod 100 comprises an airtight structure 10 comprising walls forming an enclosed interior 11 and an exposed exterior 12. To allow a passage for the one or more individual to enter the pod 100 from the exterior 12 to the interior 11 (and vice versa), at least one of the walls comprise at least one opening that is sealable by a corresponding hatch 13.

It will be understood that the airtight structure 10 of the pod 100 seals the enclosed interior 11 from an environment external to the pod 100. In this embodiment, the external environment may comprise one or more hazard, such as gas, dust, extreme heat or cold, submersion under water, lack of breathable air, chemical contamination, biological contamination, radiological contamination, nuclear contamination, low pressure environments, high pressure environments, space, or the like. Additionally, it will be appreciated that the airtight structure 10 may comprise one or more vent (not shown) coupled to a decompression system and/or an air decontamination system, wherein in use, the decompression system is utilised to increase or decrease the ambient pressure within the enclosed interior 11 of the pod 100 to ensure survivability of the individual(s) therein, and the air decontamination system is utilised to remove dangerous or undesirable airborne substances within the enclosed interior 11 of the pod 100. The air decontamination system may be of the type supplied by PlasmaShield, that is fit-for-purpose in such applications that bio-decontamination is required within medical spaces.

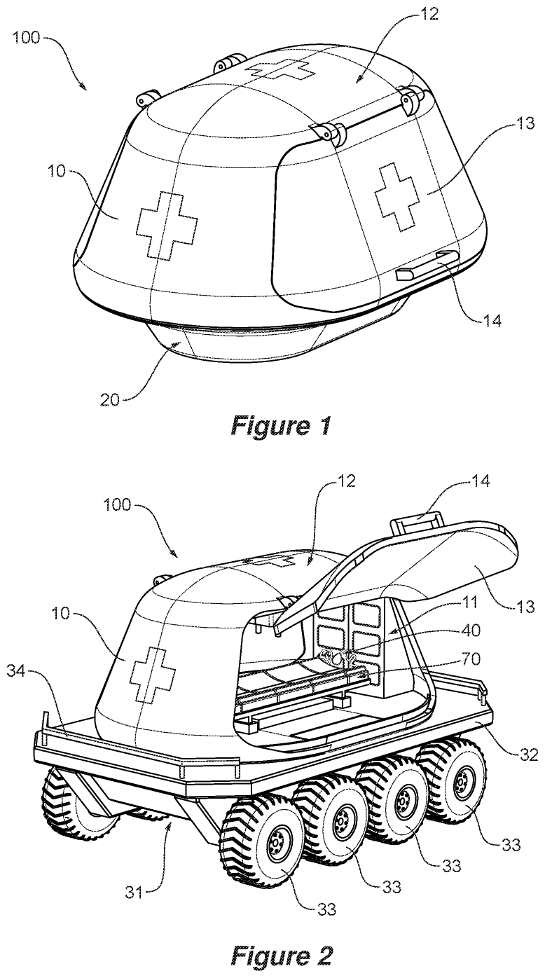

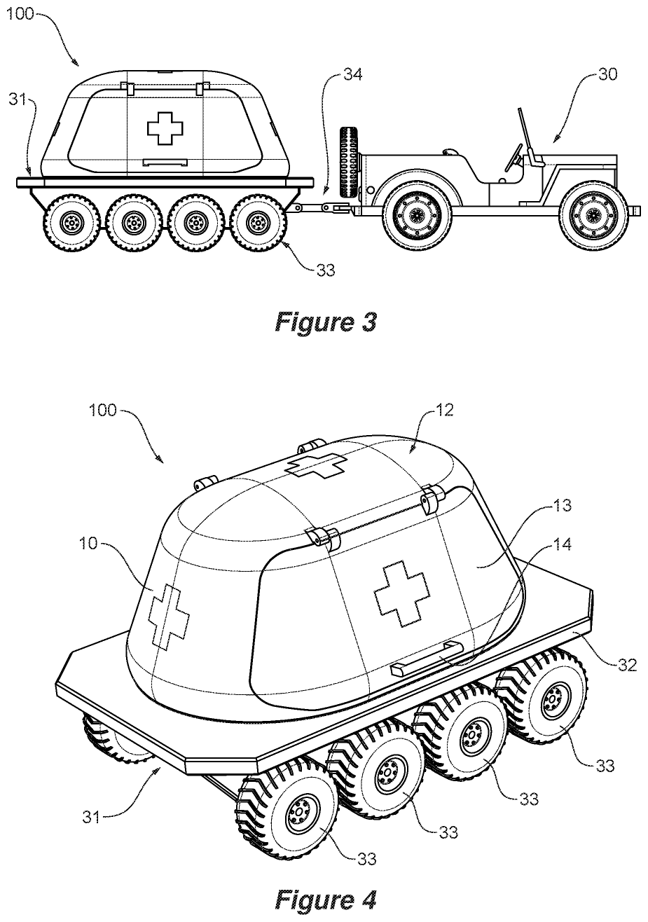

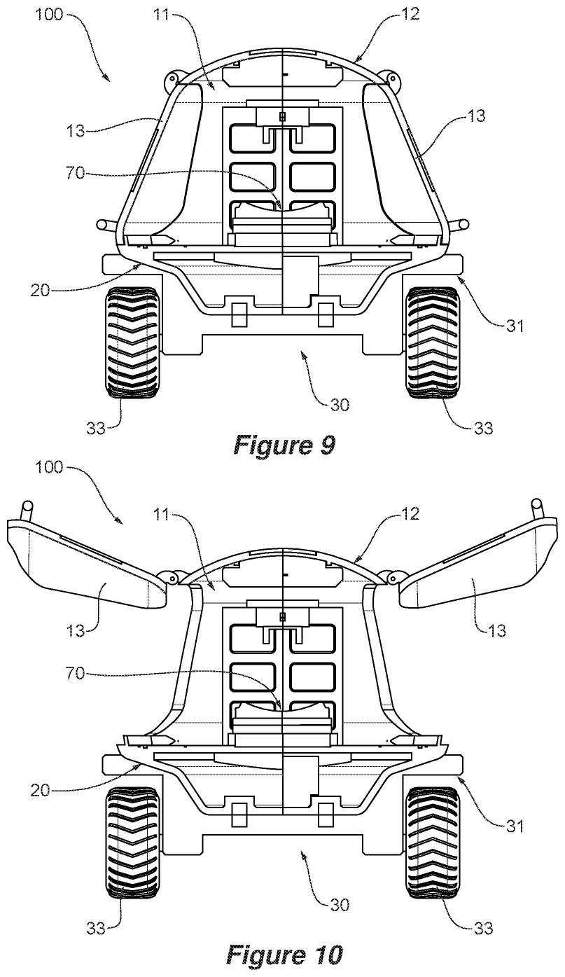

With particular reference to FIGS. 1, 2, 3, 4, 9, and 10, each hatch 13 is designed to maintain the airtight structure 10, and being sized and shaped to enable the individual(s) to enter/exit the pod 100 via the associated opening. Each hatch 13 further enables the removal or addition of equipment from the interior 11 to the exterior 12 (and vice versa) of the pod 100. Each hatch 13 may comprise a handle 14 on an exterior of the hatch 13 to enable the hatch 13 to be opened or closed from the exterior 12 of the pod 100. Each hatch 13 may further comprise a locking or securing means to enable the individual(s) within the pod 100 to secure the interior 11 of the pod 100. The locking means (not shown) may be remotely operable, by a controller or the like, to enable remote locking/unlocking of the corresponding hatch 13 in the instance that the individual(s) within the pod 100 is/are unable to operate the locking means. It will be appreciated that the or each hatch 13 may be a door that maintains the airtight structure 10, or one of the hatches 13 may be a window that allows visibility into and out of the pod 100.

Referring now to FIGS. 1, 3, 6, 8 and 9, the pod 100 may also comprise a connection means 20 on the exterior 12 of the pod 100. The connection means 20 being particularly designed so it enables the pod 100 to be connected to a vehicle 30. The vehicle 30 may be any one of a manned or unmanned vehicle 30, wherein the vehicle 30 is selected depending on the mode of transport required to relocate the pod 100 from the first location to the second location. For example, the vehicle 30 is selected to be any one of an air, water, land or space travel capable vehicle. Whereby, in use when connected to the pod 100 via the connection means 20, the manned or unmanned vehicle 30 enables the transport of the pod 100 and the individual(s) contained therein to be transported from the first location to the second location. It will be appreciated that the connection means 20 may be any form of existing connector on the exterior 12 of the pod 100 that advantageously enables the pod 100 to be connected to any external vehicle 30 capable of transporting (e.g. towing) the pod 100.

In one embodiment, the connection means 20 comprises stabilizing features, such as electromagnetic or air cushions. Whereby, in use, the stabilizing features act so as to improve the stability of the pod 100 when the connection means 20 is utilised to connect the pod 100 to the vehicle 30.

In one embodiment, the connection means 20 further comprises a reinforced undercarriage. Wherein the reinforced undercarriage is particularly designed to protect the underside of the pod 100 from damage during transportation or connection to the vehicle 30. Additionally, the reinforced undercarriage enables the pod 100 to be positioned on surfaces that may have otherwise caused damage to the pod 100 when positioned thereon.

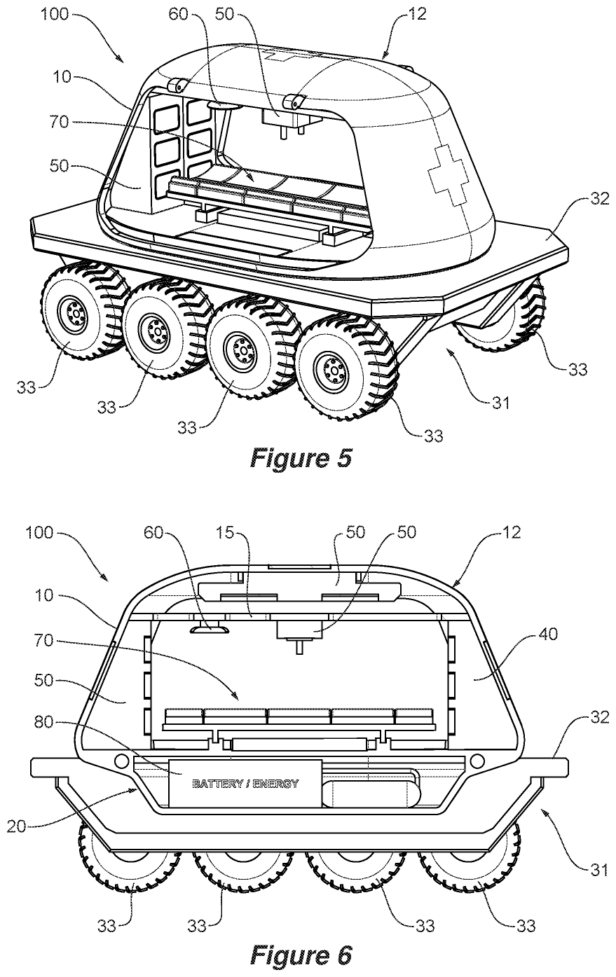

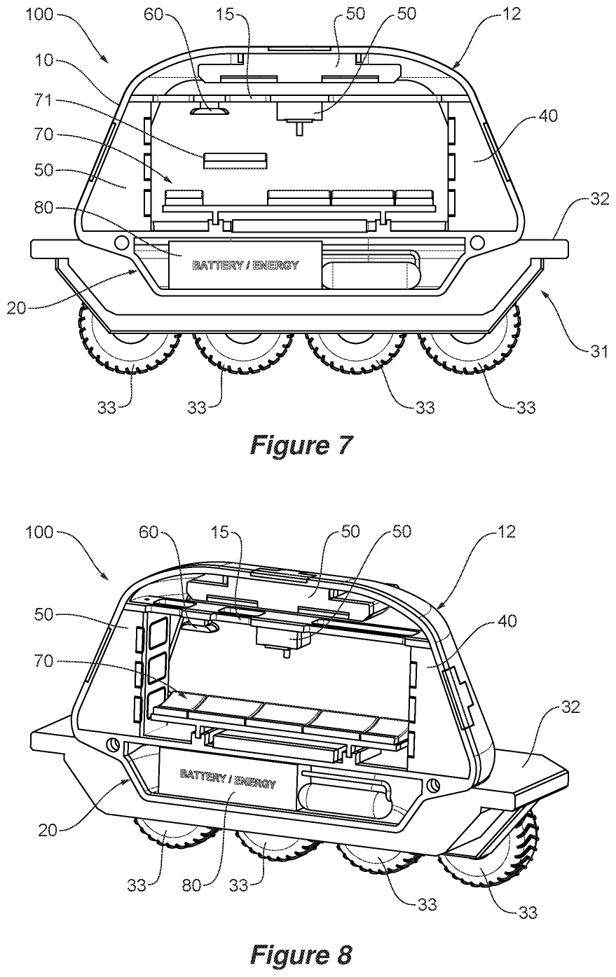

In one embodiment, particularly illustrated by FIGS. 2 to 10, the connection means 20 enables the pod 100 to be connected to a mobile platform 31. Wherein, in use, the mobile platform 31 is one that is capable of supporting and transporting the pod 100 when connected thereto. The mobile platform 31 comprises a dock 32 that is sized and shaped to receive the connection means 20, a plurality of wheels 33 or tracks on an underside of the dock 32 to enable transport of the platform 31, and one or more tethering mechanism 34 enabling the mobile platform 31 to be connected to an external vehicle 30 or allow the platform 31 to be moved by one or more individual.

In one embodiment, the connection means 20 (with or without the use of the mobile platform 31) enables the pod 100 to be connected to an external Uninhabited Underground Vehicle (UGV). The UGV may be remotely operable, and capable of transporting and retrieving the pod 100 from the first location to the second location. Examples of UGVs that may be utilised in this manner include those (but is not restricted to) by Brokk Robotics or Rheinmetall. It will be appreciated that the connection means 20 advantageously allows the pod 100 to be connected to any external transport means that permits the pod 100 to be transported and/or retrieved from the first location to the second location. It will be appreciated that the first location may be on ground, in sea, in air, or in space.

In an alternative embodiment, the connection means 20 (with or without the use of the mobile platform 31) may enable the pod 100 to be connected to an external Uninhabited Aerial Vehicle (UAV, such as a drone). The UAV may, for example, comprise a propeller-, jet-, ramjet-, or rocket-propulsion means, enabling the pod 100 to be capable of aerial/space transportation from the first location to the second location.

In yet another alternative embodiment, the connection means 20 (with or without the use of the mobile platform 31) may enable the pod 100 to be connected to an external amphibious vehicle. Wherein the amphibious vehicle has the capability of transporting the personnel transportation pod 100 on both land and water environments.

In yet another alternative embodiment, the connection means 20 (with or without the use of the mobile platform 31) may enable the pod 100 to be connected to an external uninhabited submarine vehicle. Wherein the uninhabited submarine vehicle has the capability of transporting the personnel transportation pod 100 from a first location that is a submarine environment (either military or civilian) to the second location. In this embodiment, either one of the pod 100 or the uninhabited submarine vehicle may comprise an in-built decompression chamber, for use at the first submarine environment (i.e. a subsea depth) to ensure safe extraction of the one or more individual being extracted via the pod 100.

In yet a further alternative embodiment, the connection means 20 (with or without the use of the mobile platform 31) may enable the pod 100 to be connected to an external uninhabited space travel vehicle. Wherein the uninhabited space travel vehicle (otherwise may be referred to as intra-planetary transportation, such as for lunar surface colonies, as well as inter-planetary transportation) has the capability of transporting the personnel transportation pod 100 from a first location that is in space to the second location, and the uninhabited space travel vehicle comprises ground or jet-propelled means to enable transportation of the pod 100.

The pod 100 may also comprise an integrated remote monitoring system. Wherein the remote monitoring system 40 comprises a monitoring means capable of connecting to and monitoring vital signals from the individual(s) in the pod 100. The remote monitoring system 40 also further comprises means enabling the monitored vitals of the individual(s) to be transmitted to an external receiver at a location (such as a hospital, clinic or the like), so that a medical practitioner can monitor the vital signals of each individual within the pod 100 remotely.

In one embodiment, the remote monitoring system 40 may further comprise an environmental monitoring means for monitoring the interior and/or exterior atmosphere of the pod 100. The environmental monitoring means being particularly designed so as to enable monitoring of the exterior atmosphere of the pod 100 to ensure that the interior 11 of the pod 100 is suitably maintained to enable survivability of the individual(s) therein. In this embodiment, the environmental monitoring means may also monitor the exterior atmosphere of the pod 100 to ensure that the pod 100 is operable in the first location, as this location may be an austere or hostile environment. By way of example only, and not limited to, the environmental monitoring means may monitor the exterior of the pod 100 so as to ensure the pod 100 is operable when submerged underwater, in a hazardous gas environment, in an extremely hot/cold environment, an irradiated environment, or even in the absence of breathable air (e.g. in space). The environmental monitoring means may operate with additional systems of the pod 100, such as the decompression system to increase or decrease ambient pressure in the interior 11 of the pod 100, or with the air decontamination system to remove dangerous or undesirable airborne substances within the enclosed interior 11 of the pod 100.

The pod 100 may further comprise a medical support system comprising life support equipment 50 and at least one remotely operable robot capable of manipulating equipment within the pod 100, and/or each individual. In one embodiment, the life support equipment 50 comprises medical and surgical equipment that is capable of being manipulated by the at least one remotely operable robot, or by one or more medical personnel, or by one or more individual within the pod 100. Wherein, in use, the robot is able to utilise the medical and surgical equipment to aid the one or more individual within the pod 100 and assist in sustaining their life. The one or more robot is/are remotely operable by a remote medical practitioner or the like at a location external to the pod 100, wherein the one or more robot is/are controlled via the remote monitoring system 40 of the pod 100 which transmits and receives signals to operate the one or more robot. By way of example only, and not limited to, the medical and surgical equipment may include multiple limb-and life-saving devices, such as, minor surgical kits, tourniquet, Quickstop, TXA/Fibrinogen, REBOA kit, Ultrasound, X-ray, ventilator systems, CT scanner, non-invasive monitoring systems, splints, medications, Yanker sucker, Crichoidectomy kit, Bag and mask, pathology kits/items, and Guedel tubes. It will be appreciated that other medical and surgical kits are envisaged for the pod 100 beyond those that are listed here. It will also be appreciated that the pod 100 comprises one or more refrigerated compartment (not illustrated) that is capable of storing one or more of the life-saving device or medical kit (e.g. pathological kits/items, medication, or the like) that require refrigeration.

In one embodiment, the medical support system may further comprise an oxygen supply system for providing oxygen to the interior 11 of the pod 100 and the individual(s) therein. The oxygen supply system may comprise one or more oxygen tank for storage of oxygen, and an oxygen generator for creating oxygen. It will be appreciated that the oxygen generator has the ability to produce oxygen and store the produced oxygen in the one or more tank. It is envisaged that the oxygen supply unit, and/or the oxygen generator, may further comprise means and processes that allow for the laser splitting of carbon dioxide to produce oxygen for use in the pod 100. The oxygen supply system may co-operate with the environmental monitoring means of the pod 100 so as to maintain survivability of the individual(s) within the pod 100, by adjusting oxygen levels within the pod 100. Furthermore, it will be appreciated that the environmental monitoring means may be coupled with a range of other systems of the pod 100 (such as other medical support system equipment 50) to adjust the conditions inside the pod 100, to sustain the life of the individual(s) therein. The one or more oxygen tank may be located on the pod 100 such that the one or more oxygen tank is/are readily accessible, and replenishable, during maintenance of the pod 100.

The pod 100 may further comprise a communication system. Wherein, in use, the communication system enables remote monitoring of each individual within the pod 100, communication between the one or more individual within the pod 100 and a remote medical practitioner or the like at a location external to the pod 100, and remote utilisation of each remotely operable robot to treat each individual. The communication system may comprise a multi-modal communication means 60 comprising one or more of video, voice and immersive technology-mediated communication means that enable a remote user, such as a medical professional, to virtually monitor and communicate with the individual(s) within the pod 100. Examples of multi-modal communication means 60 include multiple augmented reality cameras, with built in augmented reality tablets to enable continuous 2-way communication between an individual within the pod 100 and the remote user (e.g. an external medical professional). It will be appreciated that many modes of communication means 60 are envisaged for the pod 100, beyond those discussed herein. The purpose of the communication means 60 is to enable the remote communication with each individual within the interior 11 of the pod 100, so as to monitor visually (e.g. via cameras), verbally (e.g. integrated microphones) and virtually (e.g. augmented reality means) the condition of each individual.

In one embodiment, the pod 100 further comprises at least one support stretcher 70 in the interior 11 of the pod 100. The support stretcher 70 may be one that is removable from the interior 11 of the pod 100 to the exterior 12 via the hatch 13, and vice versa, to assist in accommodating the individual(s) within the pod 100. It will be appreciated that the pod 100 comprises one or more internal fixture that enables each support stretcher 70 to be secured to the interior 11 of the pod 100, so as to ensure the safe transport of the individual on the stretcher 70. Each support stretcher 70 may comprise a segmented base 71, wherein each segment 71 of the stretcher 70 is removable and comprises a support strap to assist in securing an individual thereto. The segmented base 71 of the stretcher 70 advantageously enables the stretcher 70 to be reconfigured into a seat (not shown). In this way, the seat may be secured to the interior 11 of the pod 100 and enable the individual to sit thereon. Additionally, in this way by being configured as a seat, the stretcher 70 utilises less space within the pod 100 and enables additional room for equipment, other individuals, or an additional stretcher 70 in a flat or seat configuration. It will also be appreciated, that by being segmented 71, the stretcher 70 may also be folded and stowed within the interior 11 of the pod 100 when not required. It will additionally be appreciated that the configuration of the one or more stretcher 70 within the pod 100 is adjusted based on the requirements of the pod 100, the pod's application, the number of individuals requiring accommodation within the pod etc. It will further be appreciated that the interior 11 of the pod 100 is modular in nature, that is to say, in place of one or more support stretcher 70 the pod may further comprise at least one integrated seat in the interior 11 of the pod 100. The configuration of the pod 100 may also be adjusted during transportation of the pod 100.

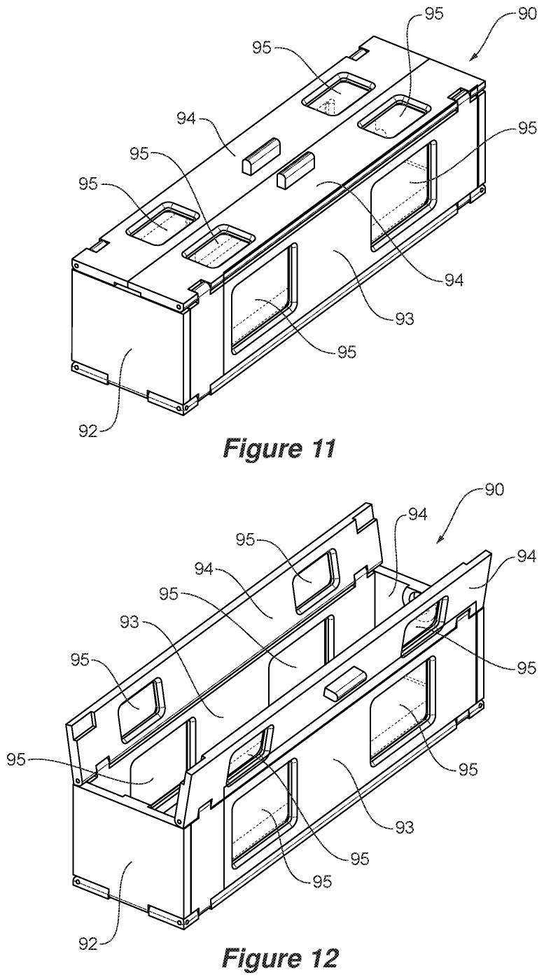

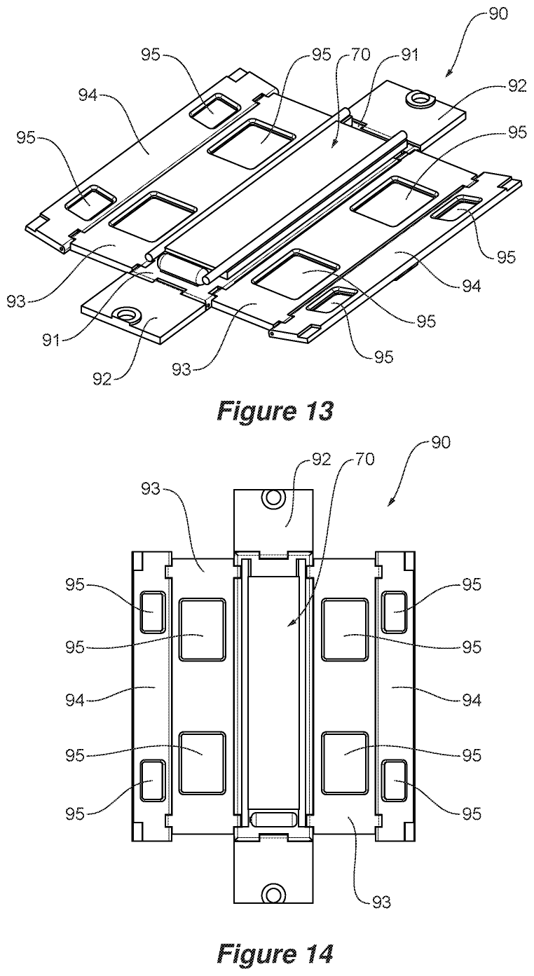

In one embodiment, illustrated in any one of FIGS. 11 to 14, the pod 100 may further comprise a modular stretcher 90 that is a system 90 that comprises the support stretcher 70. The modular stretcher 90 is separable from the pod 100, in that it is removable from the interior 11 of the pod 100 via one or more of its openings/hatches 13. The modular stretcher 90 comprises a modular, collapsible, and foldable design that enables it to be reconfigured from an assembled state (best illustrated in FIGS. 11 and 12) to a disassembled state (best illustrated in FIGS. 13 and 14). The modularity and foldable design of the modular stretcher 90 advantageously permits it to be connected within the interior 11 of the pod 100 in an arrangement where the individual on the stretcher 70 within the modular stretcher 90 is easily and readily accessible by one or more medical practitioner.

The modular stretcher 90 comprises a base 91, pair of end walls 92 and a pair of side walls 93, wherein the walls 92 and 93 extend from the base 91 and are pivotably or hingedly connected thereto. In its assembled state (FIGS. 11 and 12) the base 91, walls 92 and 93 define a housing that can receive an individual through an opening. The one or more of the walls 92 or 93 may each comprise a door 94 that enables closure of the opening of the modular stretcher 90. When the modular stretcher 90 is in its assembled state (FIGS. 11 and 12) the one or more doors 94 enables access to the housing, via the opening, to enable either or both of the stretcher 70 and the individual to be loaded or unloaded from the modular stretcher 90.

The modular stretcher 90 may comprise one or more window 95, wherein the window(s) 95 may be on any one or more of the walls 92 or 93 or doors 94. The one or more window 95 enables viewing of, and access to, the individual when within the housing of the system 90. In this way, during transportation of an individual within the system 90, one or more medical practitioner may view, access and check on the individual via the one or more window 95. It will be appreciated that the windows 95 may be considered to be ‘access points’ that enable both viewing of and access to from an exterior of the modular stretcher 90 to its interior.

Advantageously, by virtue of the hinged connection of each wall 92 and 93 to the base 91, the modular stretcher 90 is easily converted from its assembled sate to its disassembled state. This enables an individual and stretcher 70 to be easily loaded and unloaded from the modular stretcher 90, and enables the system 90 itself to be easily loaded and unloaded from the pod 100.

The modular stretcher 90 is particularly advantageous in that it can be unloaded from the pod 100 and taken to an injured individual when the individual is in a location that the pod 100 itself cannot access (such as a confined space). The modular stretcher 90, similar to the pod 100, may comprise one or more system that allow for the remote monitoring of and provide medical/life support to the individual therein, until that individual is transported from the location that the pod 100 cannot access to the pod 100 itself. The modular stretcher 90, also like the pod 100, may comprise an air tight structure when in its assembled state, wherein the walls 92 or 93 and doors 94 seal an interior of the system 90 from the environment external to the system 90.

In another embodiment, the modular stretcher 90 itself is directly connectable to the vehicle 30, wherein the vehicle 30 is any one of a manned or unmanned vehicle 30, wherein the vehicle 30 is selected depending on the mode of transport required to relocate the system 90 from the first location to the second location. For example, the vehicle 30 is selected to be any one of an air, water, land or space travel capable vehicle. In this embodiment, the modular stretcher 90 may be used independent of the pod 100 to transport the individual from the first location to the second location. It will be appreciated that this embodiment is particularly advantageous in the instances where the pod 100 itself cannot access an injured individual, or in the instance that the individual can be (or needs to be) transported straight to the second location (i.e. the safe location) without first being relocated to the pod 100.

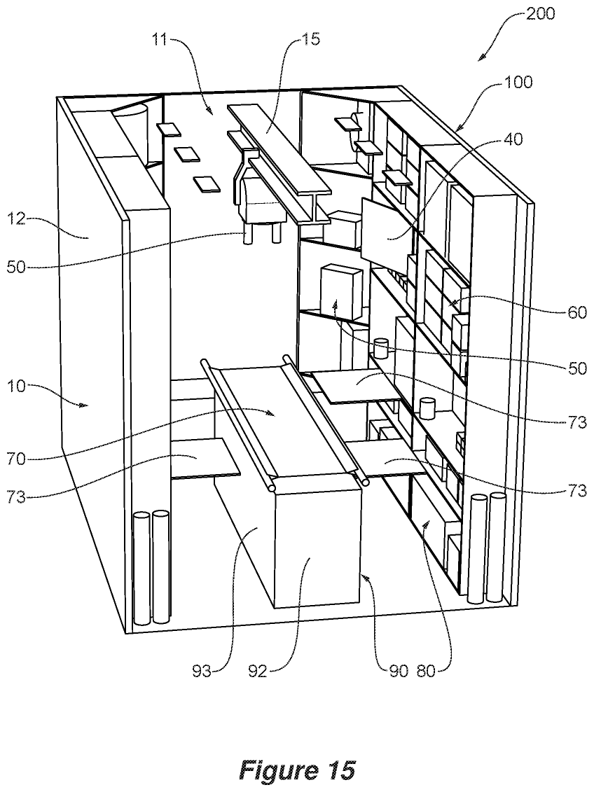

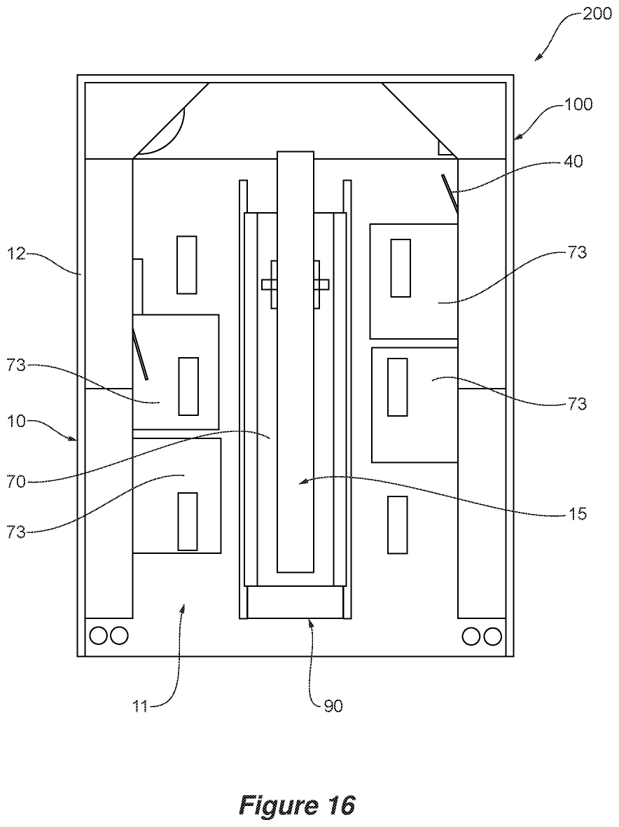

In the above embodiments, referring now to FIGS. 15 and 16, the pod 100 combined with the modular stretcher 90 form a personnel rescue system 200. The personnel rescue system 200 in these Figures illustrate an alternative embodiment of the pod 100. It will be appreciated that the pod 100 is not limited to a particular arrangement or overall shape as illustrated in any one of the disclosed Figures, and additionally, that although the Figures illustrate the pod 100 comprising a single stretcher 70 or modular stretcher 90 therein, the pod 100 or rescue system 200 can be configured to house two or more stretchers 70 or 90 as required. Wherein the personnel rescue system 200 is for transporting, remote monitoring of, and/or providing medical support to at least one individual. An exemplary use of the personnel rescue system 200 may include:
Locating the modular stretcher 90 to an initial location of an injured individual (which may be inaccessible by the pod 100);
Loading the individual onto stretcher 70 and into the modular stretcher 90 via the doors 94 thereof;
Monitoring the individual within the modular stretcher 90 via the one or more window 95 and its remote monitoring systems;
Transporting the modular stretcher 90 with the individual therein to the pod 100 which is located at the first position;
Receiving the modular stretcher 90 within the interior 11 of the pod 100 via the hatch 13, and locating the modular stretcher 90 within a desired position within the interior 11 of the pod 100;
Utilising the remote monitoring 40, medical support 50 and communication systems 60 to monitor, support and communicate with the individual;
Connecting the pod 100 to a vehicle 30 to transport the pod 100, and thus the personnel rescue system 200 to the second position (which may be a safe location, such as a hospital, clinic, base camp or the like).

A preferred embodiment of the personnel rescue system 200 is as follows. The personnel rescue system 200 is for transporting, remote monitoring of, and/or providing medical support to at least one individual. The system 200 comprises: a pod 100 (which may be the pod 100 of any one of the embodiments disclosed herein) and at least one modular stretcher 90. The pod 100 comprises: an airtight structure 10 having an enclosed interior 11, an exposed exterior 12, and at least one opening providing for providing a passage between the exterior 12 and the interior 11 and vice versa, wherein the opening is sealable by a hatch 13; a connection means 20 on the exterior 12 of the pod 100, wherein the connection means 20 enables the pod 100 to be connected to a vehicle 30; a remote monitoring system for monitoring the individual; a medical support system for stabilising, sustaining and providing medical care to the individual; and a communication system, wherein in use, the communication system enables remote monitoring of each individual within the pod and remote utilisation of the medical support system. Wherein, in use, the at least one modular stretcher 90 transports the individual from a location exterior of the pod 100 to the enclosed interior 11 of the pod 100, and the remote monitoring, medical support and communication systems are utilised to monitor, support and communicate with the individual within the pod.

In one embodiment, the exposed exterior 12 of the pod 100 comprises an external surface that is resistant to environmental conditions, such as, but not limited to, weather, lack of breathable air, hazardous gas/dust, extreme heat/cold, submersion underwater, chemical/biological/nuclear contamination etc. Additionally, the enclosed interior 11 of the pod 100 comprises an internal surface that is at least partially separated from the external surface by insulation (not shown). In this embodiment, the external and internal surfaces may be separated by thermal insulation capable of maintaining suitable conditions within the interior 11 of the pod 100 to sustain the accommodated individual(s) therein. In an alternative embodiment, the internal and external surfaces may be further separated by additional reinforcement layers to aid in providing protection to the pod 100 and the individual(s) therein. It will be appreciated that it is for this reason that the pod 100 comprises the airtight structure 10, so as to seal the interior 11 (and the accommodated individual(s)) of the pod 100 from the external environment.

In one embodiment, the external surface of the exposed exterior 12 is sufficiently reinforced to enable the pod 100 to withstand impact. In this embodiment, it will be appreciated that the external surface may be any one or more of Kevlar, Twaron, graphene, multi-walled carbon nanotubes, or similar material(s) capable of providing protection and heat-resistance to the pod. The external surface may also be selected for the pod 100 based on the application of the pod 100. For example, if the pod 100 is to be utilised in a military/combat environment, the exterior surface may be selected to be a bullet proof material. It will also be appreciated that other materials are anticipated without being listed herein. Additionally, in this embodiment, the exposed exterior 12 may also comprise means to allow attachment thereto. Wherein, in use, the means to allow attachment to the exposed exterior 12 allows attachment of additional reinforcing materials to increase the ability of the pod 100 to withstand impact. For example, the additional reinforcing materials that may be added to the exposed exterior 12 include armour, steel plates, or bullet proofing material.

In one embodiment, the pod 100 further comprises a power system 80 comprising one or more means for providing power to the pod 100 and the systems associated therewith. The one or more means for providing power to the pod 100 may transfer power derived from a number of known means, but may also be particularly derived from a source of renewable energy. The source of renewable energy may be any one of (but not limited to) solar or hydrogen energy captured and stored by known means either comprised within the pod 100, or external thereof. In the instance that the pod 100, for example, comprises one or more solar panel (not shown) thereon, the pod 100 may comprise the means to be self-powered. Advantageously, the pod 100 being capable of being powered by renewable means allows the pod 100 to have greater operability in isolated locations not proximal to conventional power sources.

In one embodiment, the pod 100 may further comprise at least one connection port for connecting the pod 100 to an external power source to charge the power system 80. The at least one connection port can also further connect the pod 100 to an additional communication system (e.g. Ethernet or data cables).

In one embodiment, the power system 80 comprises one or more battery or other storage medium that is/are either located within the interior 11 or on the exterior 12 of the pod 100. The battery(ies) or other storage medium(s) being particularly designed to store power required by the pod 100 and the systems associated therewith.

In any one of the embodiments, the pod 100 may further comprise at least one generator that is part of the power system 80. The generator may be located within the interior 11 of the pod 100, or attached to the exterior 12 of the pod 100, or it may be relocated within the interior/exterior based on a desired interior 11 configuration and use of the pod 100. The generator may be any known power generator that is capable of providing power to the pod 100 and the systems associated therewith.

In any one of the embodiments, the pod 100 may further comprise one or more overhead assembly 90 that enables mounting, attachment, and manipulation of equipment thereto. The one or more overhead assembly, in a preferred embodiment, is one or more gantry 15 arrangement allowing the mounting, attachment, and manipulation of equipment thereto. As best illustrated in FIGS. 6 and 8, which shows a single gantry 15 arrangement, the gantry 15 is attached to an internal ceiling of the pod 100, and may comprise one or more equipment attached thereto such as life support equipment 50, medical/surgical equipment, communication systems 60, or any other equipment that may preferably be located above an individual on the stretcher 70, or simply be located so that the equipment does not use floor space within the interior 11 of the pod 100. Although not illustrated, it is envisaged that there may be multiple gantries 15 within the interior 11 of the pod 100 that enable mounting, attachment, and manipulation of equipment to advantageously free up floor space within the pod 100 and enable equipment to be easily manipulated within the pod 100 by one or more medical practitioner or the one or more robot. As one example only and illustrated in FIGS. 6 and 8, the gantry 15 may comprise an x-ray machine 50 attached thereto. In this example, advantageously the x-ray machine 50 is located above the individual on the stretcher 70, where the x-ray machine 50 is able to travel along the gantry 15 to take the required images of the individual at various points on the individual as they lay on the stretcher 70. It will be appreciated that in alternate examples, it is envisaged that the gantry 15 may comprise other equipment, both medical and non-medical, that has an advantage when positioned above an individual on the stretch 70, and being manipulated within the pod 100 relative to the individual (such as CT scanners, oxygen/gas supplies, screens, support straps/braces, monitoring units/connectors, lights, cameras and others not mentioned).

It will be appreciated that in any one of the above embodiments, the pod 100 is sized and shaped to house more than one individual, and that the individuals may be utilising the pod 100 for differing reasons. For example, one individual may be injured (i.e. a patient) and another individual may be an on-site (para) medic or nurse (i.e. a medical practitioner). In this example, the pod 100 accommodates all individuals, advantageously allowing the onsite (para) medic or nurse to assist the patient with the equipment within the pod 100 to sustain their injuries/life, while simultaneously communicating and cooperating with another medical professional/practitioner (e.g. a doctor or surgeon) external to the pod via the communication means. In this way, advantageously the onsite (para) medic or nurse receives on demand and instantaneous assistance/instruction from the external medical professional to aid with the patient, while the pod 100 is able to be transported from its current (first) location by the vehicle 30 to the hospital/clinic (second) location. That is to say, in this example with the patient and (para) medic, there are advantages to both individuals to successfully treat the injured patient due to on demand and instantaneous communications with the external medical professional, and that the pod 100 is concurrently transporting both individuals to the second location.

In any one of the embodiments, the pod 100 comprises a modular, reconfigurable interior 11. That is to say, the interior 11 of the pod 100 is not limited to the configuration illustrated within the Figures, the associated systems and equipment that may be located within the pod 100 is not at ‘locked’ or ‘set’ positions and may be arranged/configured as required (e.g. the medical support system 50, the communication system 60, the remote monitoring system 40, the power system 80 and the stretchers 70, 90). It can be considered that the equipment or associated systems internal of the pod 100 is configurable such that the pod 100 is fit-for-purpose. Such as, the pod 100 may have a particular configuration when it is intended for use as a critical/intensive care unit, and it may be reconfigured for the instance that it is required as a specialist laparoscopy unit, or a dental surgery unit, or any other specialist medical unit. It will be appreciated that a number of internal configurations of the pod 100 is envisaged, as the pod 100 is intended to act as a ‘critical care unit’ (or ‘intensive care unit’ or ‘intensive therapy unit’), so long as the pod 100 is capable of acting as a transportable health care facility that enables medical personnel to care for injured individuals therein, as the injured individual is transported from the first location to the second location.

In the illustrated embodiments, the stretcher 70 is shown as being centrally located within the pod 100, however it will be appreciated that in the instances that the pod 100 is to house more than one individual that requires more than one stretcher 70, there may be two side-by-side stretchers (or any other configuration) within the pod 100. The centrally located stretcher 70 that is illustrated is one embodiment that advantageously allows the associated systems within the pod 100 to be arranged/configured about the stretcher 70, so as to best allow both the one or more medical practitioner to best manoeuvre around and access the individual on the stretcher 70 using the systems within the pod 100. The interior 11 of the pod 100 may comprise one or more seat 73 that is/are usable by the one or more medical practitioner or one or more individual that is/are not occupying the stretcher 70. The one or more seat 73 may be foldable, modular or collapsible such that it is/they are relocatable within the interior of the pod 100. In one example, the seat(s) 73 may be in a ‘stowed position’ against a wall of the interior 11, and then be placed in a ‘deployed position’ for use. The interior 11 of the pod 100 may also comprise one or more table, that is/are similar to the seat(s) 73 in that it is/they are also foldable, modular or collapsible.

It will be appreciated by those skilled in the art that the disclosed personnel transportation pod 100 has a number of applications. One such practical application may be in the mining industry, where in an underground mine, there may be one or more pod 100 located as the mine progresses underground to provide a transportable medical unit proximal to the mine face where individuals may be operating. In this scenario, should one or more individual incur injury, advantageously there is a pod 100 proximal to the individual's location that they are first in (and possibly with another person such as an onsite medic/nurse), the pod's 100 systems are utilised to stabilise and sustain injuries/life of the individual(s) while concurrently communicating their vital signs and visual of the individual to a medical professional external of the mine. Concurrently, a manned or unmanned vehicle 30 is attached to the pod 100, to transport the pod 100 and the individual(s) therein to a safe location away from the mine (such as an onsite clinic or hospital). Furthermore, the pod 100 may be loaded onto a transport vehicle such as a truck, Ute or other SUV that has means (and/or capacity) to accommodate the pod 100 thereon or therein. That is to say, the pod 100 does not necessarily require ‘attachment’ onto another vehicle, but may be transported by being loaded thereon or therein. Other practical applications (such as military/combat zones, high risk occupations such as in oil and gas drilling, remote energy sectors, forestry section, and in civilian search and rescue operations) are envisaged, and it will be understood that the pod 100 may be utilised in a similar manner to assist one or more injured individual/onsite medic/nurse. It will be appreciated, that the pod 100 advantageously increases the survivability, and reduces the severity of injuries to individual(s) by being applied in such scenarios.

Another application of the personnel transportation pod 100 is that of extra-terrestrial transportation, remote monitoring of, and/or providing medical support to one or more individual being retrieved from a location where there may hazardous conditions to humans, to a safe location where the individual(s) are able to receive care from a medical practitioner. In this application, it would be appreciated embodiments of the pod 100 are envisaged beyond those illustrated in the Figures, as the pod 100 would comprise an external and internal configuration that is suitable for survivability in extra-terrestrial environments. For example, in this application, the pod 100 may have smaller dimensions and overall payload when compared to a pod 100 that is utilised on Earth. It will be appreciated that, although the external and internal configurations may be differing to suit survivability in extra-terrestrial environments, the objective of the pod 100 to stabilise and sustain injuries/life of the individual(s) while concurrently communicating their vital signs and visual of the individual to a medical professional external of the remains the same.

It will also be appreciated that the disclosed personnel transportation pod 100 advantageously allows stranded/isolated/injured personnel to be rescued and/or retrieved from an unfamiliar/hostile first location, without the requirement of a driver for the vehicle 30 connected to the pod 100 as there is capability for the use of autonomous, remote-controlled/operated, unmanned transportation vehicles to transport the pod 100 and the individual(s) therein.

It will additionally be appreciated that the disclosed personnel transportation pod 100, when connected with the vehicle 30 for transportation, advantageously provides for a single, self-contained, self-sufficient healthcare facility that is readily transportable from a first hostile location to a second safe location, while comprising systems that permit remote monitoring of injured individuals and remotely operable robots to aid/assist the injured individuals during transport.

It will further be appreciated that the disclosed personnel transportation pod 100 can operate as a medical isolation and containment facility. Wherein the pod 100 is utilised when one or more individual have contracted an infectious agent, and require an initial point of care as they are not immediately transportable to a hospital (or other suitable medical facility). Advantageously the pod 100 does not require a large foot print to be set up, and is readily transportable when suitable for the one or more individual therein. In this way, the pod 100 may serve as additional resources to existing healthcare facilities as a “make-shift”, “mobile”, “temporary”, “transportable” or “triage” pod, before an individual therein is transferred to another suitable medical facility.

It will furthermore be appreciated that the pod 100 may be utilised similarly as existing ambulances when connected with a vehicle for transportation. The pod 100 in this way has an advantage over existing ambulances, in that the vehicle can simply disconnect from the pod 100 once it has been transported from the first location of the individual to the medical facility location. It will be understood that this advantage of the pod 100 assists to alleviate issues in healthcare systems where ambulances are required to ‘ramp’, as when the pod 100 is utilised in place of an ambulance, it comprises systems to enable remote monitoring and treatment of injured individuals within the pod 100. Accordingly, there would be less patients requiring transfer to hospitals for assessment of their condition.

A yet further advantage of the personnel transportation pod 100, in any one of the above embodiments, is that components of the pod (e.g. the airtight structure 10) may be 3D printed by existing and future technologies. In this way, the pod 100 (or at least its shell/airtight structure 10), is easily and readily manufacturable. The pod 100 then would simply be fitted with the additional systems in a modular fashion.

The reference to any prior art in this specification is not, and should not be taken as, an acknowledgement or any form of suggestion that such prior art forms part of the common general knowledge.

It will be understood that the terms “comprise” and “include” and any of their derivatives (e.g. comprises, comprising, includes, including) as used in this specification, and the claims that follow, is to be taken to be inclusive of features to which the term refers, and is not meant to exclude the presence of any additional features unless otherwise stated or implied.

In some cases, a single embodiment may, for succinctness and/or to assist in understanding the scope of the disclosure, combine multiple features. It is to be understood that in such a case, these multiple features may be provided separately (in separate embodiments), or in any other suitable combination. Alternatively, where separate features are described in separate embodiments, these separate features may be combined into a single embodiment unless otherwise stated or implied. This also applies to the claims which can be recombined in any combination. That is a claim may be amended to include a feature defined in any other claim. Further a phrase referring to “at least one of” a list of items refers to any combination of those items, including single members. As an example, “at least one of: a, b, or c” is intended to cover: a, b, c, a-b, a-c, b-c, and a-b-c.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

1. A personnel transportation pod for transporting, remote monitoring of, and/or providing medical support to at least one individual, the pod comprising:
an airtight structure comprising walls forming an enclosed interior and an exposed exterior, wherein at least one of the walls comprise at least one opening for providing a passage for the individual from the exterior of the pod to the interior and vice versa, wherein the opening is sealable by a hatch;
a connection means on the exterior of the pod, wherein the connection means enables the pod to be connected to a vehicle;
a remote monitoring system comprising a monitoring means;
a medical support system comprising life support equipment and at least one remotely operable robot capable of manipulating equipment and/or each individual; and
a communication system, wherein in use, the communication system enables remote monitoring of each individual within the pod and remote utilisation of each robot to treat each individual.

2. The personnel transportation pod of claim 1, wherein the pod further comprises at least one integrated support stretcher in the interior of the pod.

3. (canceled)

4. The personnel transportation pod of claim 2, wherein the support stretcher comprises a segmented base, wherein each segment of the stretcher is removable and comprises a support strap to assist in securing the individual(s) thereto.

5. The personnel transportation pod of claim 4, wherein the segmented base of the support stretcher enables reconfiguration of the stretcher into a seat.

6. The personnel transportation of claim 1, wherein the exposed exterior of the pod comprises an external surface that is resistant to environmental conditions, and the enclosed interior of the pod comprises an internal surface that is at least partially separated from the external surface by insulation.

7. (canceled)

8. The personnel transportation pod of claim 6, wherein the external surface is sufficiently reinforced enabling the pod to withstand impact.

9. (canceled)

10. (canceled)

11. The personnel transportation pod of claim 1, wherein the connection means enables the pod to be connected to an external uninhabited ground vehicle (UGV), wherein the UGV is remotely operable and capable of transporting and retrieving the pod from the first location, such as an isolated or hazardous location, to the second location, such as a safe location.

12. (canceled)

13. The personnel transportation pod of claim 1, wherein the connection means enables the pod to be connected to a mobile platform, whereby the mobile platform is capable of supporting and transporting the pod.

14. The personnel transportation pod of claim 13, wherein the mobile platform comprises a dock for receiving the connection means of the pod, a plurality of wheels and/or tracks on an underside of the dock to enable transport of the platform, and one or more tethering mechanism enabling the mobile platform to be connected to a UGV or a vehicle.

15. The personnel transportation pod of claim 1, wherein the medical support system further comprises an oxygen supply system for providing oxygen to the interior of the pod.

16. (canceled)

17. The personnel transportation pod of claim 1, wherein the at least one hatch maintains the airtight structure.

18. (canceled)

19. The personnel transportation pod of claim 1, wherein the remote monitoring system further comprises an environmental monitoring means for monitoring the interior and/or exterior atmosphere of the pod.

20. The personnel transportation pod of claim 19, wherein the environmental monitoring means monitors the exterior atmosphere of the pod to ensure that the interior of the pod is suitably maintained to enable survivability of the individuals therein.

21. The personnel transportation pod of claim 19 er 20, wherein the environmental monitoring means monitors the exterior atmosphere of the pod to ensure that the pod is operable in austere or hostile environments.

22. The personnel transportation pod of claim 1, wherein the life support equipment comprises medical and surgical equipment that is capable of being manipulated by the at least one remotely operable robot.

23. The personnel transportation pod of claim 1, wherein the pod further comprises a power system comprising one or more means for providing power to the pod.

24. The personnel transportation pod of claim 23, wherein the one or more means for providing power to the pod transfers power derived from a source of renewable energy to the pod.

25. The personnel transportation pod of claim 24, wherein the source of renewable energy is any one of solar or hydrogen energy stored in one or more battery or other storage medium attached to the interior of the pod.

26. (canceled)

27. (canceled)

28. The personnel transportation pod of claim 1, wherein the communication system comprises a multi-modal communication means comprising one or more of video, voice and immersive technology-mediated communication means that enable a remote user, such as a medical professional, to virtually monitor the individuals within the pod.

29. A personnel rescue system for transporting, remote monitoring of, and/or providing medical support to at least one individual, the system comprising:
a pod and at least one modular stretcher, wherein the pod comprises:
an airtight structure having an enclosed interior, an exposed exterior, and at least one opening providing for providing a passage between the exterior and the interior and vice versa, wherein the opening is sealable by a hatch;
a connection means on the exterior of the pod, wherein the connection means enables the pod to be connected to a vehicle;
a remote monitoring system for monitoring the individual;
a medical support system for stabilising, sustaining and providing medical care to the individual; and
a communication system, wherein in use, the communication system enables remote monitoring of each individual within the pod and remote utilisation of the medical support system;
and wherein, in use, the at least one modular stretcher transports the individual from a location exterior of the pod to the enclosed interior of the pod, and the remote monitoring, medical support and communication systems is utilised to monitor, support and communicate with the individual within the pod.
